(19) United States
(12) Patent Application Publication
(10) Pub. No.: US 2006/0116714 A1
(43) Pub. Date: Jun. 1, 2006

(54) Coupling and release devices and methods for their assembly and use

(76) Inventors: Ivan Sepetka, Los Altos, CA (US); Maria G. Aboytes, Palo Alto, CA (US); Ricardo Aboytes, East Palo Alto, CA (US)

Correspondence Address: WHITE & CASE LLP;PATENT DEPARTMENT, 1155 AVENUE OF THE AMERICAS, NEW YORK, NY, 10036, US

(21) Appl. No.: 11/264,402

(22) Filed: Oct. 31, 2005

Related U.S. Application Data
(63) Continuation-in-part of application No. 11/229,044, filed on Sep. 15, 2005.
Application No. 11/229,044 is a continuation-in-part of application No. 11/111,487, filed on Apr. 21, 2005.
Application No. 11/111,487 is a continuation-in-part of application No. 10/998,357, filed on Nov. 26, 2004.

(51) Int. Cl. A61M 29/00

(52) U.S. Cl. 606/200

(57) ABSTRACT

Devices and methods for coupling, delivery, detaching and controllably releasing a medical implant, and methods for the assembly and use of the devices, provide for mechanically simple and rapidly operated release of the implant without disturbing its precisely selected position. Embodiments of various aspects of the present invention employ, among other structures, a loop or other aperture-bearing engagement element releasably held by a mating retaining element, in some instances with the assistance of a member for constraining the engagement element relative to the retaining element. In another aspect, the loop or other aperture-bearing element may be at least temporarily placed in a tensile state to maintain coupling of the implant prior to release. Embodiments of the invention permit release through a practitioner actuation, such as causing one or more rotations that lead to the withdrawal of the retaining element from the loop or aperture-bearing element. Release may be immediate, or near-immediate, while the loop, which may in various embodiments be of a flexible material, is left behind with minimal or no disturbance of the implant.

### CROSS-REFERENCE TO RELATED APPLICATIONS

This application is a continuation-in-part of co-pending U.S. Ser. No. 11/______ entitled “Aneurysm Treatment Devices and Methods” filed Sep. 15, 2005 which is a continuation-in-part of co-pending U.S. Ser. No. 11/111,487, filed Apr. 21, 2005, which in turn is a continuation-in-part of co-pending U.S. Ser. No. 10/998,357, filed Nov. 26, 2004, all of which are are incorporated herein by reference in their entireties. This application is also a continuation-in-part of each of the above-identified co-pending U.S. Ser. Nos. 11/111,487 and 10/998,357.

### FIELD OF THE INVENTION

This invention relates to medical coupling and release devices and methods for their assembly and use.

### BACKGROUND OF THE INVENTION

Implants have been used in the treatment of a variety of abnormalities, conditions and diseases, particularly cardiovascular indications such as arterial atherosclerosis and aneurysms. Various mechanisms have been proposed for detachment and release of implants delivered using catheter systems or other endoluminal means. However, there remains a need for mechanisms and methods of delivery of implants, and for their controlled release, that are increasingly precise, reliable, and simple to assemble and use.

### SUMMARY OF THE INVENTION

Implant delivery devices may be used in treating a variety of endoluminal systems, such as the vascular, urinary or gastrointestinal systems of a patient. These devices are useful for delivery of implants for remediation of vascular system abnormalities and conditions, including arterial aneurisms and arterial atherosclerosis or restenosis, particularly of cardiac arteries. The implant may be of any type, including, without limitation, embolic implants and patent devices, such as stents. These implants may be referred to generally below simply as an implant, regardless of type. The present invention, in its various aspects and embodiments, provides for an implant attachment and release mechanism that permits retention of the implant during precise positioning of the implant and subsequent controlled withdrawal or release of the implant based on the need for its repositioning, or its satisfactory positioning.

Fundamentally, aspects and embodiments of the present invention recognize the use of openings, apertures, penetrations or other surfaces of genus greater than or equal to one (as such structures are referred to in topological terms) to create positively controllable and releasable locking and interlocking relationships advantageous in forming detachable couplings. Other embodiments of aspects of the present invention recognize tensile structures such as loops (or other structures of genus greater than or equal to one) as coupling and detachment mechanisms, which may act as tethers, lashes or other fasteners involving retention based on tension in the detachment mechanism. Although the illustrated embodiments show an example of such a tension loop detachment mechanism coupled to an implant device and releasable from the delivery device, this tensile structure could alternatively be coupled to the delivery device and releasable from the implant. Similarly, although the illustrated embodiment is a flexible structure, which is a structure that is intended to flex during use, and may comprise one or more filamentary elements, part or all of it could also be relatively more rigid in other embodiments of the invention. The flexible filamentary structure can have any flexible structure including without limitation, a shapeless structure, such as a floppy, string-like or spaghetti-like structure having no consistent shape in its elongate dimensions. Moreover, although these tension loops or other tensile structures according to the present invention employ tension to carry out their coupling and retention prior to detachment, and may be in tension when in use, they do not need to be in tension at all times.

In some embodiments of certain aspects of the invention, a first element having an opening (for example, but without limitation, a loop of elongate, flexible material) is provided that is anchored on one device, passes through an opening associated with another device and is retained releasably in position—keeping the two devices attached as long as the retention is positively maintained. Retention can be provided by any number of elements, but in various embodiments retention and engagement may be actuated distally to the point of retention. For endoluminal use inside a patient, a practitioner can thus actuate the retention element from outside the patient, and cause cessation of the retention of the first element having an opening (e.g., the loop) so that the two devices are no longer coupled. In some embodiments, but without limitation, an edge of an orifice in a tubular delivery device serves to maintain a position of a portion of a detachment element having an opening (e.g., a tension loop), relative to a retention member (which may be a pin or similar structure formed by the tip of a wire inserted through a delivery catheter, in certain embodiments). A variety of arrangements can embody this principle regardless of the particular geometries of the components, and which components are attached to which.

The invention, in one embodiment, provides a coupling device for use in delivery of an embolic implant by a delivery tool having a detachment feature. The coupling device comprises a loop structure coupled to the embolic implant, wherein the loop structure is configured to be releasable by the detachment feature. A loop structure, as used herein, includes a structure having an attached or integral elongate element having an aperture or opening. A detachment feature will be understood to include a structure having an attached or integral element actuatable to cause detachment of another structure or element. The detachment feature can be actuatable by the physician using the device upon delivery to release the implant.

In particular embodiments of one aspect of the delivery devices of the invention for use with an implant, the release and detachment of the implant, upon actuation of the detachment mechanism, is immediate or near immediate.

Another feature of embodiments of certain aspects of the delivery devices of the invention for use with an implant, is that once positioned in the target lumen, the implant is stably maintained in that location during the actuation of the detachment mechanism by the practitioner, causing release and detachment of the implant. In other words, the actuation of the release and detachment mechanism in no way, or only minimally, disturbs the implant and its precise positioning. This is stability of the implant after positioning is achieved because the delivery device does not recoil on actuation and subsequent detachment and release, thereby leaving the implant undisturbed in the original placement position.

In an embodiment of one of its aspects, the invention provides an engagement and detachment mechanism for a delivery device used for placement of an embolic or other implant in the patient. The implant has an associated releasable engagement feature, the releasable engagement feature having an aperture. The delivery device has an associated surface having at least one aperture adapted for receiving the engagement feature and at least a subset of its aperture, permitting positive control of engagement or detachment of the implant.

In an embodiment of another aspect, the invention provides a coupling and detachment device for use in delivery of an implant by a delivery tool having an associated actuatable detachment feature, the coupling and detachment device comprising a loop structure coupled to an implant and being configured to be coupled to the detachment feature of the delivery tool and released from the delivery tool upon actuation of the detachment feature.

An embodiment of another aspect of the invention provides a detachment mechanism for use in endoluminal placement of an implant by a delivery device, in which the implant is coupled to a releasable engagement structure provided with an opening, the detachment mechanism comprising: a surface coupled to the delivery device having at least one penetration adapted for receipt and controlled release of a portion of the engagement structure that includes the at least one opening.

In yet another embodiment of one of its aspects, the invention provides a mechanism for release of an embolic implant from a delivery device comprising: (i) a first element having an opening and associated with the embolic implant; (ii) a second element having an opening and associated with the delivery device; and (iii) a retaining member retractably passing through at least one of the openings of the first and the second elements.

In another embodiment of one of its aspects, the invention provides a detachment device for an endoluminal implant comprising a loop, in which a capability to maintain tension in the loop prevents detachment and a cessation of the capability to maintain tension in the loop permits detachment.

Another embodiment of an aspect of the invention provides a detachment apparatus for controlled release of an implant using a delivery device, at least one of the implant and delivery device having an associated surface defining a first aperture and having an associated retaining member. The detachment apparatus comprises an engagement member coupled to a first one of the implant and the delivery device and also comprises a second aperture. The second one of the implant and delivery device includes the associated surface defining the first aperture. The engagement member is adapted to pass through the first aperture to releasably engage the retaining member. The engagement member can be a structure that includes a loop or other aperture, and the retaining member can be a structure having an attached or integral element that in one position prevents detachment, and in another position permits detachment and release.

In one embodiment of another aspect, the invention provides a detachment apparatus for an implant delivery device, the apparatus being adapted for use in releasably engaging a detachment structure. The detachment structure is coupled to the implant and is provided with an aperture. The detachment apparatus comprises: (i) a retaining member adapted to engage the aperture of the detachment structure coupled to the implant, when the retaining member is in a first position, and to disengage the aperture of the detachment structure when the retaining member is in a second position; and (ii) a constraining member positioned relative to the retaining member for at least partially maintaining a spatial relationship between the detachment structure and the retaining member. The detachment apparatus can include a loop or a retainer or both. The constraining member can be a structure having an attached or integral element that limits the degrees of freedom of motion of another element. For instance, in one example, the constraining member can be an edge of an opening or aperture (through which the detachment structure may be positioned while retained) such as an opening in a wall of a tubular element.

In yet another embodiment of one of its aspects, the invention provides a method for assembling a detachment mechanism for an endoluminal implant for placement by a delivery device, the method comprising the steps of: (i) providing a detachable engagement structure having a first opening and adapted to pass through an opening associated with the delivery device; and (ii) coupling the detachable engagement structure to the implant.

In another embodiment of one of its aspects, the invention provides a method for assembling a detachment mechanism for an endoluminal implant for placement by a delivery device, the method comprising the steps of: (i) providing a loop structure configured to maintain attachment of the implant to the delivery device when placed tension, and to detach the implant from the delivery device upon cessation of a capability to maintain tension; and (ii) coupling the loop structure to at least one of the implant and the delivery device.

In another embodiment of one of its aspects, the invention provides a method for assembling an endoluminal implant delivery and detachment system comprising the steps of: (i) providing an implant having a tension loop releasable detachment component; (ii) providing a delivery device having a tension loop releasable retention component; and (iii) coupling the tension loop releasable detachment component to the tension loop releasable retention component.

In one embodiment of a particular aspect, the invention provides a method for delivering an endoluminal implant by a delivery and detachment system. The system includes a detachable engagement structure having a first opening and is adapted to pass through an opening associated with the delivery device to be retained by an actuatable retaining member. The detachable engagement structure is coupled to the implant. The method includes the following steps: (i) positioning the implant; and (ii) actuating the retaining member to cease retaining and to release the engagement structure; thereby detaching the implant. In one embodiment the detachment of the implant occurs immediately upon actuation of the retaining member.

In one embodiment of another particular aspect, the invention provides a method for sensing the positioning and release of an endoluminal implant from a delivery and detachment system. The system includes a detachable engagement structure having a first opening and adapted to pass through an opening associated with the delivery device, and the detachable engagement structure being coupled to the implant. The method includes the following steps: (i) sensing the position of each of the engagement structure and the implant prior to detachment of the engagement structure; (ii) sensing the position of each of the engagement structure and the implant after detachment of the engagement structure; and (iii) determining that the position of the implant is separated from the position of the engagement structure after detachment of the engagement structure. In one embodiment the sensing includes sensing of a radiopaque marker on the implant and a radiopaque marker on the engagement structure. In another embodiment a detectable marker is located on or near an engagement structure, such as the distal tip of a delivery catheter or pusher tube, and also on a functional wire (for instance, a wire that functions as a retaining member). Actuation of the retaining member causes a change in position of the retaining member relative to the distal tips of a delivery catheter, and/or the pusher tube. This change in relative positions of the markers can be sensed and may be visualized, for instance by fluoroscopy, demonstrating that the detachment and release has occurred.

In an embodiment of another particular aspect, the invention provides a coupling and detachment device for use in delivery of an implant by a delivery tool. The delivery tool has an associated actuatable detachment feature, and the coupling and detachment device includes a flexible loop structure coupled to the implant. The flexible loop structure is configured to be coupled to the detachment feature of the delivery tool and released from the delivery tool upon actuation of the detachment feature.

In another embodiment of another particular aspect, the invention provides a mechanism for release of an implant from a delivery device. The mechanism includes a flexible first element having an opening and associated with the implant; a second element that has an opening and is associated with the delivery device; and a retaining member retractably passing through at least one of the openings of the first and the second elements.

### BRIEF DESCRIPTION OF THE DRAWINGS

The invention can be more readily understood with reference to the following embodiments and the examples depicted in the figures, which are not to scale.

FIG. 1A shows an implant with an attached loop disposed along a portion of the outer surface of a pusher member and threading through an aperture in the outer wall of the pusher member, the aperture of the loop receiving the distal tip of a control wire disposed within the pusher member, all in accordance with an embodiment of the present invention. The pusher member is threaded to receive a reciprocal threading of the control wire.

FIG. 1B shows the detached implant released by rotating the control wire to release the threaded connection between it and the pusher member and withdrawing the control wire from the loop, in accordance with an embodiment of the present invention.

FIG. 2A shows a coil implant with an attached loop disposed as in FIG. 1A, above. The pusher member is likewise threaded to receive a reciprocal threading of the control wire, in accordance with an embodiment of the present invention; however, the control wire in this embodiment extends beyond the distal end of the pusher member and engages the implant coil.

FIG. 2B shows the detached implant coil similarly released, in accordance with an embodiment of the present invention, by rotating the control wire to release the threaded connection between it and the pusher member and withdrawing the control wire from the implant coil and the loop.

FIG. 3A shows, in an embodiment of the present invention, a coil implant with an attached loop penetrating an aperture in the distal tip of a pusher member, the aperture of the loop receiving the distal tip of a control wire disposed within the pusher member. The pusher member is likewise threaded to receive a reciprocal threading of the control wire; however, the control wire in this embodiment is engaged by an aperture in and terminates at the distal tip of the pusher member.

FIG. 3B shows the detached implant coil similarly released, in accordance with the present invention, by rotating the control wire to release the threaded connection between it and the pusher member and withdrawing the control wire from the implant coil and the loop.

FIG. 3C shows an end view of the distal tip of the pusher member showing the aperture for receiving the loop and a second aperture for engaging the distal tip of the control wire.

FIG. 4A shows a flexible implant loaded in and secured to a delivery catheter in accordance with an embodiment of the present invention.

FIG. 4B shows the flexible implant still secured to and partially discharged from the delivery catheter, in accordance with an embodiment of the present invention.

FIG. 4C shows the flexible implant released and detached from the delivery catheter, in accordance with an embodiment of the present invention.

FIG. 5A shows a knotted flexible implant loaded in and secured to a catheter by a wire passing through several loops attached to the implant.

FIG. 5B shows the discharged flexible implant, in accordance with an embodiment of the present invention.

FIG. 6A shows a control wire passing through a hypotube (the needle tube of a hypodermic needle) fitted with a female portion of a luer lock. In this embodiment of the invention, the proximal end of the control wire is affixed to a male portion of the luer lock.

FIG. 6B shows the position of the control wire of FIG. 6A having been advanced distally, permitting mating of the male and female portions of the luer fitting and locking the control wire in place at the proximal end in accordance with this embodiment of the invention.

### DETAILED DESCRIPTION

In its various embodiments and aspects, the present invention makes possible detachable coupling mechanisms for endoluminal and other medical delivery that maintain reliable closure, are simple of structure and assembly, and that permit positive control and detachment or disengagement of the delivered implant or other object from the delivery device.

As used in this specification, terms like loop, opening, pass-through, aperture, orifice or hole generally refer to topological surfaces of genus one or greater than one, the genus of a topological surface being the maximum number of cuts along closed simple curves that can be made without separating a section of the surface. Thus, a solid sphere or block has a topological genus of zero, since any cut along a closed curve of its surface separates the enclosed area of the surface. A hollow cylinder has a topological genus of one, since one cut along a closed curve in its surface in the direction of the cylindrical axis opens the walls of the cylinder, but does not separate any area of its surface. In a classic example, a doughnut and a coffee mug with a handle are both examples of structures of genus one. Similarly, surfaces of higher genus number encompass openings of that higher number.

The term “detachment,” such as when the term is used in conjunction “mechanism,” “means,” “device,” or other label, is used to refer at least to an ability to decouple, but is not intended to exclude structures that can be both decoupled in one action, and separately detached, or be released in a subsequent step.

In particular aspects and embodiments, the present invention provides coupling by a non-friction penetration interlock of an element of an implant and an element of a delivery device. As used herein the term “non-friction interlock” denotes an interlock that does not particularly require friction between the interlocking components for proper function, at least as compared with other forces, though it will be understood that the interlock may not necessarily be altogether friction-free. In some instances, tension loop structures according to the present invention may be embodiments of non-friction interlock detachment mechanisms of the present invention.

In another particular embodiment, the present invention provides a delivery system (also referred to as a delivery device) for an implant, such as a vascular occlusion device, the implant having a proximal end and a distal end, the distal end having a engagement element coupled to it, comprising: (i) an introducer component having a longitudinally extending lumen or cannula and proximal and distal ends; (ii) a pusher component slideable within the introducer component, the pusher component having a distal end positioned adjacent to the distal end of the implant; and (iii) a core component having a distal end and extending through the pusher component and parallel to the implant so that the distal end of the core component contacts the engagement element, thereby applying a tensile force to the implant.

The invention, in one embodiment of its aspects, provides for a tension loop detachment mechanism, which can act as a tether, lash or other fastener providing retention based on tension in the detachment mechanism. Although the embodiments illustrated in the drawings show the tension loop detachment mechanism coupled to the implant device and releasable from the delivery device, it will be understood that alternative embodiments are within the scope of the present invention. For instance, in another embodiment of one aspect, the tension loop detachment mechanism can be coupled to the delivery device and releasable from the implant. Similarly, although the illustrated embodiments relate to a flexible structure comprising one or more filamentary elements, part or all of the tension loop detachment mechanism can be relatively more rigid in other embodiments of the invention.

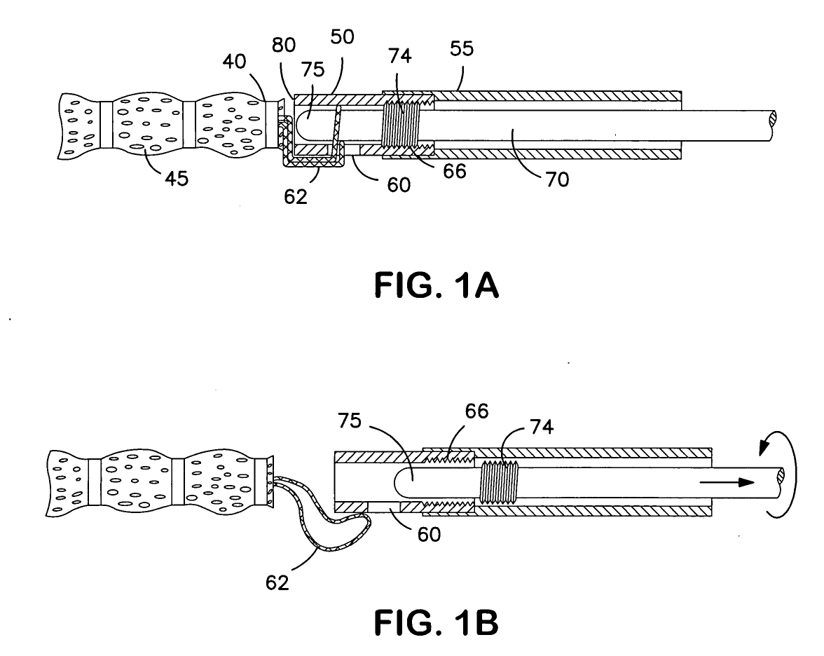

FIG. 1A shows one embodiment of a connection between a proximal end 40 of implant 45 at the distal end member 50 of pusher member 55 of a delivery device. Distal end member 50 comprises a lateral opening 60 to receive loop member 62 attached to implant 45 and has a threaded proximal inner bore 66. A portion of the distal end of wire 70 has reciprocal threads 74 that engage threaded bore 66. In the engaged position of the embodiment shown in FIG. 1A, the distal end 75 of wire 70 terminates at a position level with the distal orifice 80 of distal end pusher member 50 and penetrates loop member 62, engaging implant 45. The assembly shown in FIG. 1A including the entire length of the implant 45 is housed within a microcatheter.

Pusher member 55 is advanced distally to expel implant 45 through the microcatheter. In the event that implant 45 is incorrectly placed, pusher member 55 is retracted proximally to reinsert implant 45 into the microcatheter, during which loop member 62 may be under a tensile load while maintaining implant 45 attached to the delivery device. This controlled delivery or retraction of implant 45 into the target lumen can be repeated until the desired positioning of implant 45 is achieved. When implant 45 is appropriately positioned wire, 70 is rotated causing disengagement of wire 70 from threading 66 and permitting wire 70 to be retracted. Wire 70 is retracted sufficiently to withdraw its distal end 75 beyond lateral opening 60 as shown in FIG. 1B, such that it no longer penetrates loop member 62. Wire 70 is thereby disengaged from loop member 62 and from pusher member 50. Loop member 62 is no longer restrained by wire 70 and is free to pass through lateral opening 60, which had constrained loop member 62, releasing implant 45 from any attachment to the delivery device. Loop member 62, which may, here and elsewhere in this specification, be referred to as a loop, a tensile loop or a tensile element, is one embodiment of an engagement member having a surface with an aperture (the hole in the loop), capable of being retained by wire 70, which is, in turn, an embodiment of an engagement or retention member. Lateral opening 60 is an embodiment of an aperture in a surface associated with, or coupled to, an embodiment of one aspect of the delivery device, that may operate as a constraining member bearing on loop member 62, keeping it from disengaging wire 70.

Distal end pusher member 50 and the distal end 75 of wire 70, in certain embodiments, may each comprise a marker radiopaque material, such as platinum, to assist an practitioner during delivery. When distal end pusher member 50 and distal end 75 of wire 70 are in the engaged position, the radiopaque markers will be visible as a single spot under fluoroscopy; however, when distal end pusher member 50 and distal end 75 of wire 70 disengage, and release the loop member 62 from the implant 45, the separation of the markers will be visible as two separate spots under fluoroscopy indicating release of the implant 45.

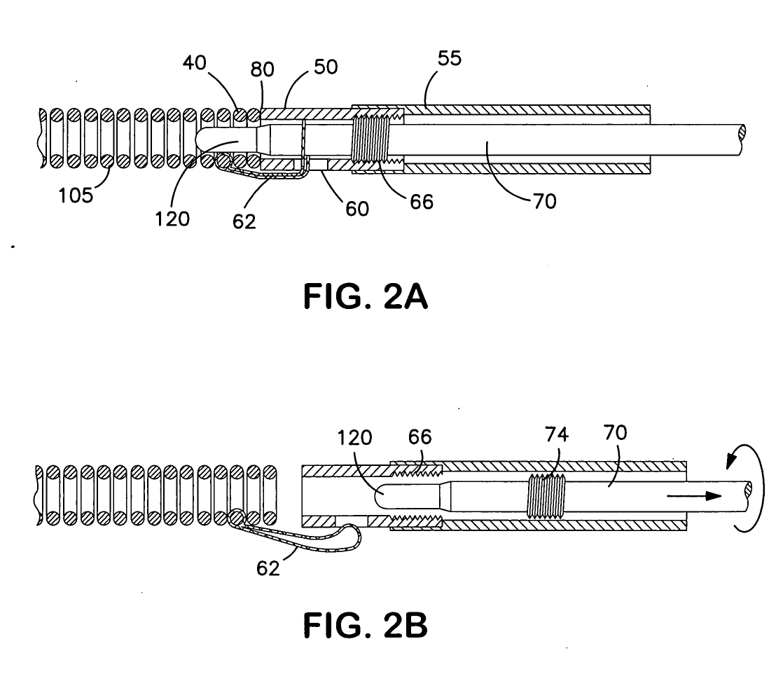

FIG. 2A depicts another embodiment of the connection between the proximal end 40 of a coiled implant 105 and the distal end member 50 of the pusher member 55. The features of this delivery device are essentially identical to those of the delivery device depicted in FIG. 1A, except that the distal end protrusion 120 of wire 70 in this embodiment protrudes beyond the distal orifice 80 of distal end pusher member 50 extending into the interior of the proximal end 40 of coiled implant 105. The tip or protrusion 120 of the distal end of wire 70 may be adapted to be received into the coil. For instance, the protrusion 120 of distal end of wire 70 can be of a smaller diameter than the remainder of wire 70 as shown in FIG. 2A.

Implant 105 is released, in an embodiment of the invention, by rotation and withdrawal of wire 70 in a proximal direction to a position such that protrusion 120 of distal end 75 is withdrawn beyond lateral opening 60 and no longer penetrates loop member 62. Loop member 62 is no longer restrained by wire 70 and is free to pass through lateral opening 60 releasing implant 105 from any attachment to the delivery device as shown in FIG. 2B.

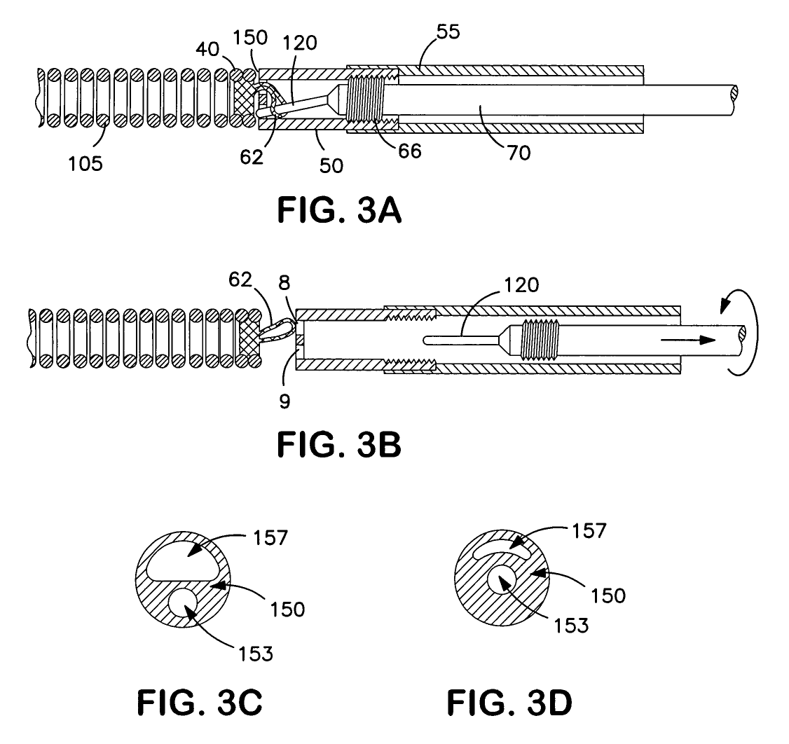

FIG. 3A depicts yet another embodiment of the connection between the proximal end 40 of a coiled implant 105 and the distal end member 50 of the pusher member 55. The features of this delivery device are essentially identical to those of the delivery device depicted in FIG. 2A, except that the distal end protrusion 120 of wire 70 in this embodiment protrudes into a first opening 153 (in this case a circular opening) of two openings in the end cap or distal face 150 of distal end member 50 of the pusher member 55. The loop member 62 attached to implant 105 passes through the second opening 157 (in this case in the shape of a segment of a circle) in distal face 150 of distal end member 50 shown in FIG. 3C.

Implant 105 is released, essentially as before, by rotation and withdrawal of wire 70 in a proximal direction to a position such that angled or deformable flexible, pivotable or otherwise radially moveable protrusion 120 of distal end 75 is withdrawn from opening 153 and and no longer penetrates loop member 62. Loop member 62 is no longer restrained by wire 70 and is free to pass through opening 157 in distal face 150, detaching and releasing implant 105 as shown in FIG. 3B.

In a further embodiment openings 153 and 157 in distal face 150 of distal end member 50 are shaped such that opening 153 is axially located in distal face 150 to receive distal end protrusion 120 without any angular deformation. In this embodiment, opening 157 is an elongate channel confined to an off-center location as shown in FIG. 3D.

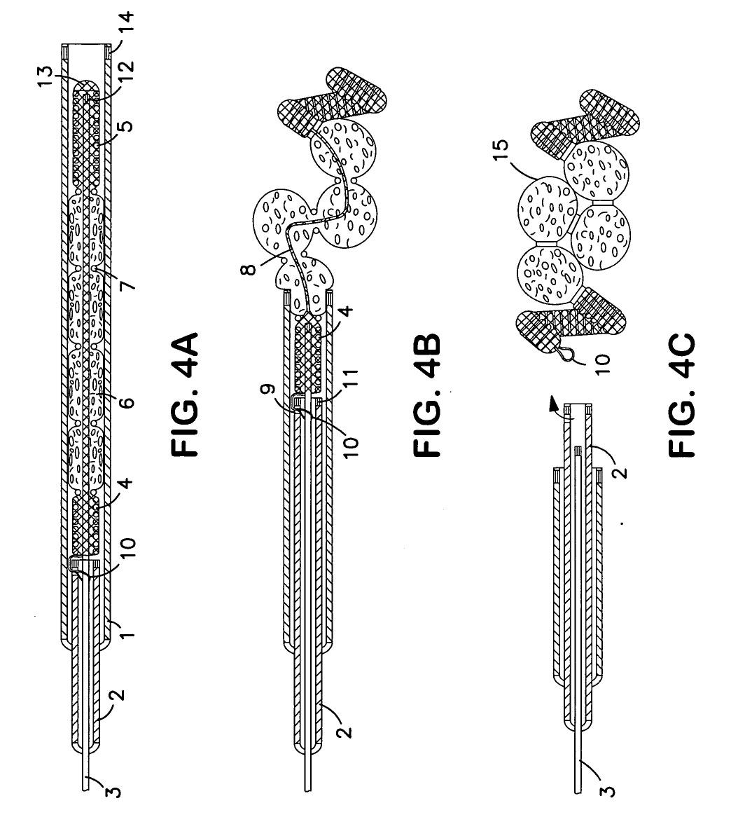

FIG. 4A shows details of implant loading and coupling during placement with a delivery system of a particular embodiment of the invention. Delivery catheter 1 has a distal tip 14 through which the implant is deployed. Pusher tube 2 disposed within the delivery catheter 1 has a proximal tip 11 and side hole 9. An internal core wire 3 has a distal tip 12 and extends distally into the pusher tubing and has a proximal end extending beyond the proximal ends of the pusher tube 2 and the delivery catheter 1. The implant optionally comprises a proximal memory coil 4 and a distal memory coil 5 that can be configured from a platinum or polymer braided jacket 8 sheathing the distal end of the core wire 3. A loop member 10 is coupled to the proximal end of memory coils 4 and 5. Side hole 9 in pusher tubing 2 is of a size and placement suitable to receive loop member 10, which is attached to the end of the proximal memory coil 4.

One or more of the following components can be provided with a marker for sensing and visualization during placement, deployment and detachment of the implant from the delivery device: the distal tip 14 of the delivery catheter, the proximal tip 11 of the pusher tube 2, the distal tip 12 of internal core wire 3, can each carry a marker for visualization, such as a radiopaque marker, for monitoring its location by fluoroscopy.

The distal tip 14 of the delivery catheter is guided by the practitioner to the desired location. Once accurately positioned, the implant is deployed as shown in FIG. 4B by gently forcing the pusher tube 2 distally relative to the distal tip 14 of the delivery catheter, thus pushing the implant off the distal end of core wire 3 and out of the delivery catheter. Frictional resistance between the pusher tube 2 and the delivery catheter can be reduced by injection of a physiologically compatible fluid into the lumen between the pusher tube and the delivery catheter. Finally, the core wire 3, serving as a retention member, is moved in a proximal direction relative to the pusher tube 2 sufficiently to withdraw from the loop member 10, serving as an engagement structure, attached to the proximal coil 4, detaching the implant from the delivery device as shown in FIG. 4C.

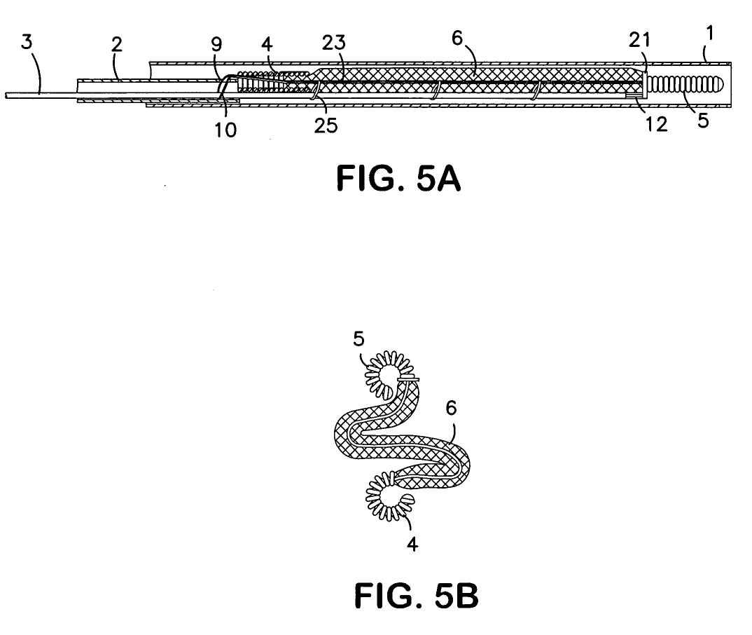

FIG. 5A shows details of implant loading and coupling during placement with a microcatheter delivery system of another particular embodiment of the invention used in cranial access and embolization. In this embodiment, a soft segmented implant 6 reinforced only with sutures is loaded into the delivery catheter 1, preferably a microcatheter. Proximal and distal platinum coils 4 and 5, respectively with helical shape memory can be fused or attached at the ends of the implant. This type of soft implant 6 is suitable for embolization of a vessel or aneurysm, for example, by cranial access. The implant is abutted by a coaxial tubular pusher sheath 2, sheathing an internal core wire 3 extending distally parallel with the major axis of the implant to provide the push during delivery through the microcatheter 1 and also support during deployment or withdrawal and repositioning if needed. The core wire 3 may optionally comprise a radiopaque tip for visualization. A key hole 9 is located at the distal end of pusher sheath through which passes a loop member 10. The distal tip of core wire 3 is compressed against a distal washer 21 to keep implant 6 at the required tension during the advancement to the distal part of the catheter. This tension and support is particularly important during the advancement stage of deployment when the implant might buckle or collapse, but for the rigidity of the core wire 3 to which it is harnessed. Loop member 10 is locked in place by core wire 3 extending distally in parallel with the implant and passing through the opening of the loop. Once the tip of the implant is advanced to the tip of the microcatheter 1, the core wire 3 must be retracted back into coaxial pusher sheath 2 for few centimeters (2-5 cm) and the core 3 wire and pusher 2 in combination is then used to push only the implant out of the microcatheter 1 and into the target lumen. For such flexible implants longer than 5 cm, the process is repeated whereby the core wire 3 is retracted back into the coaxial pusher sheath 2 for 2-5 cm, and the core wire-sheath construct is then used to push only the implant out of the microcatheter 1 and into the target lumen. This “inchworm” process is repeated until the entire length of the implant is delivered into the lumen. Core wire 3 must always remain within the catheter lumen and gradually retracted back into pusher sheath 2 until entire implant is discharged from the catheter 1 and positioned for the controlled detachment. Another function of the core wire 3 is to execute controlled detachment of the implant. When detachment is desired, the core wire 3 is retracted in a proximal direction to a location proximal to the side hole 9 and to release the loop member 10 and separates from the pusher sheath 2. Core wire 3 is an embodiment of a retention member creating a non-friction interlock with an aperture-bearing engagement element, loop member 10.

FIG. 5B shows a fully discharged and released soft implant with highly flexible end coils 4 and 5 for anchoring and preventing migration once the implant is discharged and released into the target lumen. The end coils 4 and 5 can be radiopaque for ease of visualization by fluoroscopy.

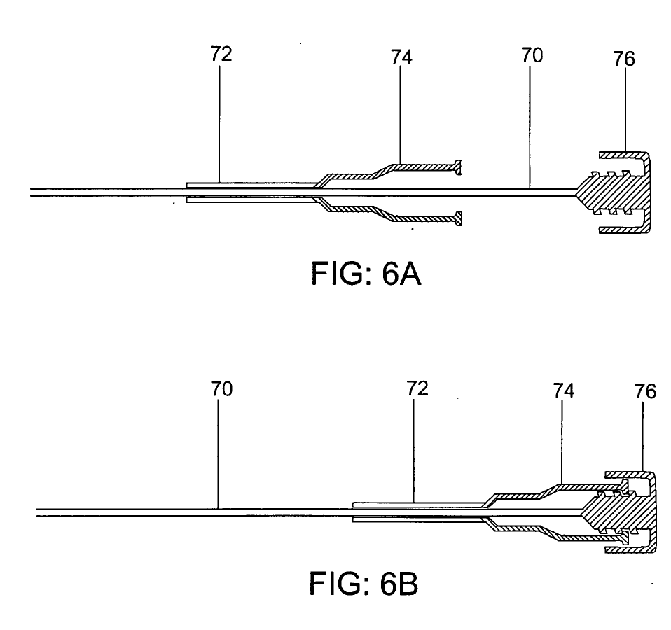

In a particular embodiment, the control wire or other selective protrusion for engaging an element having an opening or aperture, is reciprocally threaded and mated with the threading on an inside wall of the end piece of the pusher tube or other engagement element as described above. In this embodiment, the control wire passes through the lumen of a hypotube (the needle tube of a hypodermic needle) and through the male portion of a luer lock fitting attached to the hypotube 72 as shown in FIG. 6A. The proximal end of the control wire 70 can be attached or affixed to the female portion 74 of a luer lock fitting to provide rotational control of the control wire by the practitioner. The length of the control wire 70 and disposition of the threaded distal end and of the luer lock fittings can be arranged such that when the male portion 76 of the luer lock fitting is fully mated with the female portion 74, the threading of the control wire is also mated with the threading on the inner wall of the end piece of the pusher tube. The control wire 70 is thus locked in this position as shown in FIG. 6B. When the luer lock female portion 74 is turned relative to the male portion 76 to unlock the luer fitting, the female portion attached to the control wire 70 can now be rotated as necessary to unscrew the mated threads at the distal end of the wire. When the control wire 70 and the end piece of the pusher tube are no longer held together by the mated threads, the control wire can be withdrawn in a proximal direction to disengage the loop or other engagement member. Release of the engagement member detaches the coupled implant and permits its release. Release of the implant is immediate, near immediate, or at least, rapid.

In another embodiment of the invention, a delivery system further includes an interlocking wire, also referred to as a control wire, having a distal end extending longitudinally into a pusher member. The occlusion device has an engagement/release element at its proximal end, and the distal end of the pusher component has an opening through which the release element extends. The distal end of the interlocking wire is releasably held within the distal end of the pusher member, and the distal end of the interlocking wire releasably engages the release element so that the distal end of the pusher component releasably engages the proximal end of the occlusion device.

In the interlocking wire embodiment of the non-friction releasable interlock mechanism of the invention, the wire can be of any suitable material. The wire can be any substantially flexible wire having no memory set, such as a metallic wire, for instance, a NiTinol or stainless steel wire, although a hard polymer material can also be used. The interlocking wire may be of any suitable length. NiTinol may be particularly suitable because of its super-elastic properties and its ability to return to a straight conformation and not take on a set even after navigating through a tortuous path, such as may be found in vasculature. In one embodiment the interlocking wire is a NiTinol wire of from about 180 to about 220 cm in length. The interlocking wire may include a distal ground portion with a serial stepwise reduction in diameter to provide a gradual decrease in stiffness from the stiffer proximal end to the softer, more flexible distal tip. In one embodiment the serial stepwise reduction in diameter extends from about 30 cm to about 50 cm from the distal tip.

In a particular embodiment, the interlocking wire has a diameter of between about 0.014 inch and about 0.016 inch at the proximal end and has one or more stepwise reductions in diameter along its length. For example, the outside diameters at the stepwise reductions may be to about 0.010 inch in a first step, to about 0.007 inch in a second step, to about 0.005 inch in a third step, and finally to about 0.003 inch or even to about 0.002 inch at the distal tip. In one embodiment, the proximal end of the wire passes through a hypotube. The hypotube can be fitted with a luer lock that can be used to attach the hypotube to any other device, such as a side arm for receiving the control wire or other selective protrusion, or a syringe for flushing the hypotube and connected intralumenal space with a physiologically compatible fluid to reduce friction between elements of the delivery device as described above.

In certain embodiments of a delivery system of the invention, the releasable engagement element can include at least one loop. The loop can be formed from any material, including flexible materials, such as a suture material. When suture material is used, it can be formed from any flexible biocompatible material, such as for instance, a monofilament or multifilament surgical suture material, a microbraided wire, or a flexible polymer material microbraided with a platinum wire. In a preferred embodiment, the loop is formed from a 7.0 or 9.0 gauge surgical suture material (from Genzyme Medical, Boston, Mass., or from Ethicon, Somerville, N.J.).

In certain other embodiments of a delivery system of the invention, the engagement element includes a tensioning element that is restrained in a first coupled position and released in a second position. The tensioning element can be under tension in the first coupled position. In one embodiment, the tensioning element is formed into at least one loop. In another embodiment, the tensioning element is formed into a loop that is penetrated by a restraining element such as a mandrel, a control wire (also referred to as an interlocking wire), pin or other structure capable of forming an interlock with the engagement element.

In another embodiment the invention provides a mechanism for detaching an implant from a delivery device, the implant having a proximal end and a coupling component at its proximal end, comprising: (i) an engagement element coupled at a distal end of the delivery device, the engagement element having a first, engaged position and a second, disengaged position; and (ii) an energy transfer component coupled to the engagement element at a distal portion of the component to actuate the engagement element. The engagement element, when actuated, engages the coupling component of the implant when in the first position and releases the coupling component when in the second position.

In another embodiment of a mechanism of the invention, the coupling component of the implant comprises a flexible structure. In another embodiment of a mechanism of the invention, the flexible structure comprises at least one opening through which an embodiment of one aspect of the engagement element of the delivery device may pass when in the first, engaged position. In another embodiment of a mechanism of the invention, the flexible structure comprises a loop.

In another embodiment of a mechanism of the invention, the engagement element comprises a structure that moves, along an axis, from the first position to the second position. In a particular embodiment, the engagement element comprises a substantially rigid element, such as for instance a metallic wire. The metallic wire can comprise one or more metal components, such as an alloy, for instance, in one embodiment, the metallic wire engagement element comprises NiTinol.

In another embodiment of a mechanism of the invention, the delivery device comprises at least one of the group consisting of a wire and a sheath, the axis is parallel to the longitudinal axis of the delivery device, and the energy transfer component comprises at least one of the wire and the sheath. The sheath (interchangeably referred herein to as a pusher tube) can comprise any suitable sheath material, such as a hypotube. In a particular embodiment, the hypotube can comprise a thermoplastic polymer, such as for instance a PEEK (polyetheretherketone) polymer, or any other thermoplastic polymer of similar desirable physical and chemical properties. The desirable physical and chemical properties of PEEK include its high performance metal-like rigidity and resistance to wear and corrosion. In one embodiment, the distal tip of the pusher tube has an end piece, also referred to herein as an end cap or tip. In one embodiment the end piece comprises a metal, which can be a radiopaque metal such as platinum. In a particular embodiment the platinum end piece is about 1-2 mm in length and includes a threaded inside wall. The threaded inside wall can have from about 2 to about 5 rotations, and in a particular embodiment can be formed by a platinum wire coil affixed to the inside wall. The platinum wire coil can be slightly open to permit mating by a platinum wire coil of slightly smaller diameter. The platinum wire coil of slightly smaller diameter can be affixed to the engagement element as a reciprocal thread for mating with the thread of the inside wall of the end piece. In a first position, the threaded of the inside wall of the end piece is mated with the wire coil affixed to the engagement element and is locked in place. In a second position, achieved after rotating a sufficient number of turns, the threaded of the inside wall of the end piece is free from the wire coil affixed to the engagement element. In this position, the engagement element is no longer constrained by the end piece from sliding within the pusher tube. The engagement element is thereby released and can be withdrawn from the pusher tube.

In other embodiments, the end piece mating with the engagement element can include both non-frictional and frictional fastening mechanisms, including snap-lock, lock and key, a bayonet coupling, clasp, or other mechanism, rather than mated threads on the end piece inside wall and the engagement element as described above.

In another embodiment of a mechanism of the invention, the delivery device comprises a sheath and the energy transfer component comprises a wire, and the engagement element transitions between the first position and the second position as a result of a relative rotation of the wire engagement element with respect to the delivery device sheath.

In another embodiment of a mechanism of the invention, the engagement element comprises a distal portion of the wire, the coupling component of the implant comprises a loop structure, and, in the first position of the engagement element, the loop structure is stably retained about a distal portion of the wire and, in the second position of the engagement element, the loop structure is released over a free distal end of the wire.

In another embodiment of a mechanism of the invention, the distal portion of the wire has threads that engage mating threads coupled to the sheath, the delivery device comprises a distal portion having a side wall with an aperture through which the loop structure passes and is held in place when the engagement element is in the first position, and when the engagement element is in the second position, the distal end of the wire is proximal of the aperture, releasing the loop structure and allowing it to exit through the aperture.

In another embodiment of a mechanism of the invention, the control wire or other actuatable engagement element of the delivery device is operable by a practitioner.

Other objects, advantages and embodiments of the various aspects of the present invention will be apparent to those who are skilled in the field of the invention and are within the scope of the appended claims. For example, but without limitation, structural or functional elements might be rearranged, or method steps reordered, consistent with the present invention. Similarly, principles according to the present invention, and systems and methods that embody them, could be applied to other examples, which, even if not specifically described here in detail, would nevertheless be within the scope of the appended claims.

We claim:

1. A coupling and detachment device for use in delivery of an embolic implant by a delivery tool having an associated actuatable detachment feature, the coupling and detachment device comprising a loop structure coupled to the embolic implant and being configured to be coupled to the detachment feature of the delivery tool and released from the delivery tool upon actuation of the detachment feature.

2. The coupling and detachment device according to claim 1, wherein the loop structure comprises a flexible structure.

3. The coupling and detachment device according to claim 1, wherein the loop structure comprises a filamentary element.

4. The coupling and detachment device according to claim 3, wherein the filamentary element comprises a suture material.

5. The coupling and detachment device according to claim 4, wherein the suture material comprises a braided material.

6. The coupling and detachment device according to claim 2, wherein the flexible structure comprises a tension loop.

7. A detachment mechanism for use in endoluminal placement of an implant by a delivery device, in which the implant is coupled to a releasable engagement structure provided with an opening, the detachment mechanism comprising:
a surface coupled to the delivery device having at least one penetration adapted for receipt and controlled release of a portion of the engagement structure that includes the at least one opening.

8. The detachment mechanism of claim 7, further comprising an interlocking element configured for retention and controlled release of the engagement structure of the delivery device.

9. The detachment mechanism of claim 7, in which the retention of the engagement structure is effected by the interlocking element selectively protruding through the opening in the engagement structure and the release is effected by the interlocking element selectively ceasing to protrude through the opening in the engagement structure.

10. The detachment mechanism of claim 8, wherein the interlocking element comprises a distal portion of a wire carried by a lumen of the delivery device.

11. The detachment mechanism of claim 10, in which selective protrusion and cessation of protrusion of the interlocking element is effected via actuation of a proximal portion of the wire.

12. The detachment mechanism of claim 11, in which actuation of the proximal portion of the wire comprises rotation.

13. The detachment mechanism of claim 7, in which the surface coupled to the delivery device comprises an end cap of the delivery device, the delivery device having a side wall, and wherein the penetration is formed within the side wall.

14. The detachment mechanism of claim 7, in which the surface coupled to the delivery device comprises an end cap of the delivery device, and the penetration is formed within the end cap.

15. A mechanism for release of an embolic implant from a delivery device comprising:
a first element having an opening and associated with the embolic implant;
a second element having an opening and associated with the delivery device;
and a retaining member retractably passing through at least one of the openings of the first and the second elements.

16. The mechanism according to claim 15, in which the first element penetrates a second of the first and second openings.

17. The mechanism according to claim 15, in which the retaining member is configured to pass retractably through the opening of the first element.

18. The mechanism according to claim 15, in which the second element comprises a portion of the delivery device.

19. The mechanism according to claim 15, in which the second element comprises a portion of the embolic implant.

20. The mechanism according to claim 15, in which the second element is coupled to the embolic implant.

21. The mechanism according to claim 15, in which:
the first element is coupled to the embolic implant;
the delivery device comprises a tubular element having a lumen and the retaining member comprises an elongate element slidably disposed within the lumen; and
the retaining member being releasably couplable to the tubular element.

22. The mechanism according to claim 21, wherein the releasable coupling of the retaining member to the tubular element is a direct coupling.

23. The mechanism according to claim 22, wherein the direct coupling comprises threading on the retaining member engageable with complementary threading on the tubular element.

24. The mechanism according to claim 23, wherein the releasable coupling of the retaining member to the tubular element is an indirect coupling via a component coupled to the tubular element.

25. The mechanism according to claim 24, wherein the indirect coupling comprises threading on the retaining member engageable with complementary threading on the component coupled to the tubular element.

26. A detachment device for an endoluminal implant comprising a loop, in which a capability to maintain tension in the loop prevents detachment and a cessation of the capability to maintain tension in the loop permits detachment.

27. The detachment device of claim 26, in which the loop is continuously tensioned until detached.

28. The detachment device of claim 26, in which the loop is coupled to the implant.

29. The detachment device according to claim 26, in which the loop comprises a flexible filament.

30. The detachment device according to claim 26, in which the flexible filament comprises a suture material.

31. The detachment device according to claim 30, in which the suture material comprises a braided material.

32. The detachment device according to claim 27, in which the capability to maintain tension is provided in part by an edge of an aperture formed in a device for delivering the implant, in which the edge of the aperture fixes a portion of the loop at a first location relative to a reference location.

33. The detachment device according to claim 28, in which the capability to maintain tension is provided in part by a portion of a wire borne in a lumen of a device for delivering the implant, the portion of the wire protruding through the loop in order to impart tension to the loop.

34. The detachment device according to claim 27, in which a capability to maintain tension is provided in part by an edge of an aperture formed in a device for delivering the implant in combination with a portion of a wire borne in a lumen of a device for delivering the implant.

35. A detachment apparatus for controlled release of an implant using a delivery device, at least one of the implant and delivery device having an associated surface defining a first aperture and having an associated retaining member, the detachment apparatus comprising:
an engagement member coupled to a first one of the implant and the delivery device and comprising a second aperture, where the second one of the implant and delivery device has the associated surface defining the first aperture,
the engagement member being adapted to pass through the first aperture to releasably engage a retaining member.

36. The detachment apparatus according to claim 35, in which the first one of the implant and the delivery device comprises the implant, the second one comprises the delivery device, and the retaining member is associated with the delivery device.

37. The detachment apparatus according to claim 36, in which the engagement member comprises a flexible element.

38. The detachment apparatus according to claim 37, in which the flexible element comprises a filamentary element.

39. The detachment apparatus according to claim 37, in which the second aperture comprises a loop formed by the flexible element.

40. The detachment apparatus according to claim 38, in which the flexible element comprises a suture material coupled to the implant and the loop comprises the suture material.

41. The detachment apparatus according to claim 40, in which the flexible element comprises braided filament microstrands, braided platinum wire, or braided filament microstrands and platinum wire braided together.

42. The detachment apparatus according to claim 40, in which the flexible element comprises radiopaque material.

43. The detachment apparatus according to claim 40, in which the flexible element comprises a shapeless loop.

44. The detachment apparatus according to claim 35, in which the element is integral to a portion of the implant.

45. The detachment apparatus according to claim 36, in which the delivery device comprises a wall defining a lumen and the surface having an aperture comprises a portion of the wall.

46. The detachment apparatus according to claim 36, in which the delivery device comprises a wall defining a lumen and a distal end cap, and in which the surface having an aperture comprises a portion of the end cap.

47. The detachment apparatus according to claim 36, in which the retaining member comprises a wire.

48. The detachment apparatus according to claim 46, in which the surface comprises a second aperture in the end cap for receiving the retaining member.

49. The detachment apparatus according to claim 48, in which the second aperture for receiving the retaining member is offset from the center of the end cap.

50. The detachment apparatus according to claim 48, in which the first aperture comprises an elongate channel.

51. A detachment apparatus for an implant delivery device, the apparatus adapted for use in releasably engaging a detachment structure, the detachment structure coupled to the implant and provided with an aperture, the detachment apparatus comprising:
a retaining member adapted to engage the aperture of the detachment structure coupled to the implant, when the retaining member is in a first position, and to disengage the aperture of the detachment structure when the retaining member is in a second position; and
a constraining member positioned relative to the retaining member for at least partially maintaining a spatial relationship between the detachment structure and the retaining member.

52. The detachment apparatus according to claim 51, in which a transition of the retaining member between the first position and the second position comprises a translation.

53. The detachment apparatus according to claim 51, in which a transition of the retaining member between the first position and the second position comprises a rotation.

54. The detachment apparatus according to claim 51, in which a transition of the retaining member between the first position and the second position comprises a translation and a rotation.

55. The detachment apparatus according to claim 51, in which the retaining member comprises a feature for coupling the member to the device.

56. The detachment apparatus according to claim 55, in which the feature for coupling the member to the delivery device is configured to establish the coupling when the retaining member is in the first position.

57. The detachment apparatus according to claim 56 in which the feature for coupling the member to the delivery device comprises threading for engaging with complementary threading provided on the delivery device.

58. The detachment apparatus according to claim 51, wherein the retaining member comprises an elongate element and the delivery device comprises a tubular structure, the elongate element disposed within the tubular structure when the retaining member is in the first position.

59. The detachment apparatus according to 58, in which the retaining member comprises a feature for coupling the member to the delivery device, the feature for coupling the member to the delivery device being configured to establish the coupling when the retaining member is in the first position.

60. The detachment apparatus according to claim 56 in which the feature for coupling the member to the delivery device comprises threading for engaging with complementary threading provided on the delivery device.

61. A method for assembling a detachment mechanism for an endoluminal implant for placement by a delivery device, the method comprising the steps of:
providing a detachable engagement structure having a first opening and adapted to pass through an opening associated with the delivery device; and
coupling the detachable engagement structure to the implant.

62. The method of claim 61, in which the detachable engagement structure comprises a flexible filament.

63. The method of claim 62, in which the flexible filament comprises a suture material.

64. A method for assembling a detachment mechanism for an endoluminal implant for placement by a delivery device, the method comprising the steps of:
providing a loop structure configured to maintain attachment of the implant to the delivery device when placed tension, and to detach the implant from the delivery device upon cessation of a capability to maintain tension; and
coupling the loop structure to at least one of the implant and the delivery device.

65. The method according to 64, in which the step of coupling the loop structure to at least one of the implant and the delivery device comprises the step of coupling the loop to the implant.

66. A method for assembling an endoluminal implant delivery and detachment system comprising the steps of:
providing an implant having a tension loop releasable detachment component;
providing a delivery device having a tension loop releasable retention component; and
coupling the tension loop releasable detachment component to the tension loop releasable retention component.

67. The method according to claim 66, in which the tension loop releasable detachment component comprises a flexible filamentary element.

68. The method according to claim 66, in which the tension loop releasable retention component comprises a first loop retention member.

69. The method according to claim 68, in which the tension loop releasable retention component comprises a second loop retention member.

70. The method according to claim 69, in which the first loop retention member comprises an element for protruding through the tension loop.

71. The method according to claim 70, in which the second loop retention member comprises an opening in a surface of the delivery device, through which the tension loop passes, for fixing the tension loop relative to the element for protruding through the tension loop.

72. The method according to claim 71, comprising the further steps of passing the tension loop through the opening in the surface of the delivery device and engaging the portion of the tension loop that has passed through the opening in the surface of the delivery device with the element for protruding through the tension loop.

73. The method according to claim 72, in which the element for protruding through the tension loop comprises a wire borne in a lumen of the delivery device.

74. The method according to claim 73, in which the wire is coupled to a mechanism for fixing the wire relative to the delivery device.

75. The method according to claim 74, comprising the further step of releasably fixing the wire relative to the delivery device in order to retain the tension loop.

76. A method for delivering an endoluminal implant by a delivery and detachment system comprising a detachable engagement structure having a first opening and adapted to pass through an opening associated with the delivery device to be retained by an actuatable retaining member, the detachable engagement structure being coupled to the implant, the method comprising the steps of:
(i) positioning the implant; and
(ii) actuating the retaining member to cease retaining and to release the engagement structure, thereby detaching the implant.

77. The method according to claim 76, wherein, upon actuation of the retaining member, the detachment of the implant occurs immediately.

78. A method for sensing the positioning and release of an endoluminal implant from a delivery and detachment system comprising a detachable engagement structure having a first opening and adapted to pass through an opening associated with the delivery device, the detachable engagement structure being coupled to the implant, the method comprising the steps of:
(i) sensing the position of each of the engagement structure and the implant prior to detachment of the engagement structure;
(ii) sensing the position of each of the engagement structure and the implant after detachment of the engagement structure; and
(iii) determining that the position of the implant is separated from the position of the engagement structure after detachment of the engagement structure.

79. The method of claim 78, wherein the sensing is sensing of a radiopaque marker on the implant and a radiopaque marker on the engagement element.

80. A coupling and detachment device for use in delivery of an implant by a delivery tool having an associated actuatable detachment feature, the coupling and detachment device comprising a flexible loop structure coupled to the implant and being configured to be coupled to the detachment feature of the delivery tool and released from the delivery tool upon actuation of the detachment feature.

81. The coupling and detachment device according to claim 80, wherein the loop structure comprises a filamentary element.

82. The coupling and detachment device according to claim 81, wherein the filamentary element comprises a suture material.

83. The coupling and detachment device according to claim 82, wherein the suture material comprises a braided material.

84. The coupling and detachment device according to claim 80, wherein the flexible loop comprises a tension loop.

85. A mechanism for release of an implant from a delivery device comprising:
A flexible first element having an opening and associated with the implant;
a second element having an opening and associated with the delivery device;
and a retaining member retractably passing through at least one of the openings of the first and the second elements.

86. The mechanism according to claim 85, in which the first element penetrates a second of the first and second openings.

87. The mechanism according to claim 85, in which the retaining member is configured to pass retractably through the opening of the first element.

88. The mechanism according to claim 85, in which the second element comprises a portion of the delivery device.

89. The mechanism according to claim 85, in which the second element comprises a portion of the implant.

90. The mechanism according to claim 85, in which the second element is coupled to the implant.

91. The mechanism according to claim 85, in which:
the first element is coupled to the implant;
the delivery device comprises a tubular element having a lumen and the retaining member comprises an elongate element slidably disposed within the lumen; and
the retaining member being releasably couplable to the tubular element.

92. The mechanism according to claim 91, wherein the releasable coupling of the retaining member to the tubular element is a direct coupling.

93. The mechanism according to claim 92, wherein the direct coupling comprises threading on the retaining member engageable with complementary threading on the tubular element.

94. The mechanism according to claim 93, wherein the releasable coupling of the retaining member to the tubular element is an indirect coupling via a component coupled to the tubular element.

95. The mechanism according to claim 94, wherein the indirect coupling comprises threading on the retaining member engageable with complementary threading on the component coupled to the tubular element.
